(19) United States
(12) Patent Application Publication
(10) Pub. No.: US 2003/0069318 A1
(43) Pub. Date: Apr. 10, 2003

(54) Salts of analgesic substances in oil, and methods of making and using the same

(76) Inventors: Wenbin Dang, Ellicott City, MD (US); Stephen Dordunoo, Baltimore, MD (US); Abdul Kader, Perry Hall, MD (US)

Correspondence Address: FOLEY HOAG LLP, PATENT GROUP, WORLD TRADE CENTER WEST, 155 SEAPORT BOULEVARD, BOSTON, MA, 02110-2600, US

(21) Appl. No.: 09/934,250

(22) Filed: Aug. 21, 2001

(51) Int. Cl. A61K 47/00; A61K 35/78; A61K 31/137

(52) U.S. Cl. 514/786; 424/731; 424/755; 424/769; 424/757; 424/750; 424/727; 424/776; 514/649

(57) ABSTRACT

The present invention relates in part to compositions comprising salts of an analgesic agent and an oil, and methods of using and making the same. In certain embodiments, the compositions may be used as part of a treatment regimen to alleviate pain.

### INTRODUCTION

In order to administer a drug or substance such as an analgesic for extended periods, clinicians often administer such drugs through a catheter or syringe to a site where the pain is to be blocked. This method of treatment requires repeated administration when the pain is to be blocked for more than a short period of time, e.g., for more than one day. The analgesic is typically administered as a bolus or through an indwelling catheter connected to an infusion pump or by multiple injections. These methods have the disadvantage of potentially causing irreversible damage to nerves or surrounding tissues due to fluctuations in concentration and high levels of anesthetic and repeated injections. The therapeutic effect of the analgesia rarely lasts for longer than six to twelve hours, more typically four to six hours. In the case of a pump, the infusion lines are difficult to position and secure, the patient has limited, encumbered mobility and, when the patient is a small child or mentally impaired, may accidentally disengage the pump.

Pharmaceutical compositions that exhibit therapeutic effects over an extended period of time could potentially provide for treatment for longer periods of time than may be achieved by other means of administration, such as a bolus injection or topical administration of analgesic alone. Such compositions may address certain of the failings of other means of administering analgesic substances identified above and otherwise known to those of skill in the art.

In part, the present invention is directed to a pharmaceutical formulation that permits administration of the salt of an analgesic substance such that the agent achieves a therapeutic effect over an extended period of time. Certain subject compositions comprise a salt of an analgesic agent incorporated into an oil.

### SUMMARY OF THE INVENTION

In part, the present invention is directed to compositions comprising an oil and a salt of an analgesic substance, methods for treatment using the subject compositions, and methods for making and using the same. For example, one subject composition includes lidocaine HCl or an analog thereof in sesame oil. It has been learned that administration of such a composition results in a therapeutic effect of the analgesic substance for a longer time period than is observed for other modes of administration of the analgesic substance without the oil. This result is surprising in that the use of a salt of an analgesic substance in the oil, without any other delivery agent for extended release (such as those biocompatible and optionally biodegradable polymers known in the art), gives an extended therapeutic effect. Exemplary subject composition, and methods of making and using the same, are set forth in the claims appended hereto, which are hereby incorporated by this reference in their entirety into this Summary of the Invention.

The subject compositions, and methods of making and using the same, achieve a number of desirable results and features, one or more of which (if any) may be present in any particular embodiment of the present invention: (i) a single dose of a subject composition may achieve the desired therapeutically beneficial response over an extended period of time ; (ii) therapeutic effects indicative of sustained or controlled release of the analgesic substance (as its salt or in hydrolyzed form) from an oil; (iii) novel treatment regimens using the subject compositions for longer therapeutic effects from an analgesic substance; (iv) high levels of loading (by weight), e.g. greater than 1% and up to 50% or more, of a salt of an analgesic substance in oil; (v) inclusion of other therapeutic agents, including another analgesic, as a salt or otherwise, in addition to a salt of an analgesic substance, and (vi) other advantages known to those of skill in the art.

A range of analgesics, and pharmaceutically acceptable salts thereof, are contemplated by the present invention. For example, the salt of an analgesic agent may be the salt of an opiate agonist or antagonist, such as morphine sulfate, the salt of synthetic piperidine analgesic, such as fentanyl citrate, salts of non-opiate analgesics such as ketoamine HCl, salts of anti-inflammatory drugs, such as ketoprofin Na, and salts of local analgesics, such as lidocaine HCl. Further salts of analgesic agents are described in more detail below. All different pharmaceutically acceptable salts of the analgesic agents are contemplated by this invention.

In certain embodiments, the particle size of the analgesic salt may be varied. For example, the particle size of the analgesic salt may be less than 150 μm, less than 100 μm, less than 75 μm, less than 50 μm, less than 25 μm, or even less than 10 μm.

The subject compositions include an oil that is non-polar and hydrophobic. In certain embodiments, the pharmaceutically acceptable salts of analgesics are sparingly soluble, slightly insoluble, very slightly insoluble, or practically insoluble in the composition containing the oil. In certain embodiments, the oil may be chosen such that its non-polar and hydrophobic properties are matched to the solubility of the salt of the anesthetic so that the salt of anesthetic is at most only sparingly soluble in the oil and/or the resulting composition (which may contain other materials, such as other therapeutic agents). A variety of oils may be used in the present invention, as described in more detail below.

In certain embodiments, the viscosity of the oil may be varied. For example, an oil may be chosen such that the viscosity of the oil is below about 140 cSt at 20 ° C., or below about 90 at 20° C. In certain embodiments, the oil may chosen such that the viscosity of the oil is above about 20 cSt at 20° C., or above about 45 cSt at 20° C. In certain other embodiments, the polarity of the oil may be varied. For example, the oil may be chosen such that the dielectric constant is below about 20, 10, or 5.

In certain embodiments, the subject oils may be biocompatible, biodegradable or both. In still other embodiments, a percentage of the subject composition on a weight basis has less than 1%, 3%, 5%, 7% or 10% of a hydrophilic solvent.

In certain embodiments, a large percentage of the subject composition on a weight basis may be the salt of the analgesic agent. High loading levels of an analgesic agent allows a smaller amount of the subject compositions to be used for treatment with the same or greater or longer resulting therapeutic effect. For example, the salt of an analgesic substance may comprise 1 to 50% or more of the subject composition, e.g., at least 1%, at least 5%, at least 20%, at least 25%, at least 30%, at least 40% at least 60%, at least 90% or more of the composition.

In certain embodiments, other materials may be encapsulated in the subject oil in addition to the salt of an analgesic agent to alter the physical and chemical properties of the resulting composition, including for example, the profile of the therapeutic affect observed upon administration. Examples of such materials include biocompatible plasticizers, delivery agents, fillers and the like.

In still other embodiments, other therapeutic agents may be incorporated into the subject compositions in addition to the analgesic agent. The therapeutic agent may be more or less soluble or insoluble in the oil. Certain of these therapeutic agents may add to, extend other otherwise augment in some fashion the therapeutic effect of the analgesic agent. An example of such an agent is an augmenting agent, which is described in greater detail below. Other therapeutic agents may be incorporated for their therapeutic affect alone. Any of these therapeutic agents may, but are not required to, exhibit extended therapeutic effects akin to those observed for the analgesic agents.

The present invention provides a number of methods of making the subject compositions. In part, the subject invention is directed to preparation of formulations comprising an analgesic salt agent, such as lidocaine HCl, in an oil.

In another aspect, the subject compositions may be used to treat a patient or subject, such as a human. In certain embodiments, the subject compositions may be administered by injection to a subject. In other embodiments, the subject compositions are administered subcutaneously or intramuscularly.

In certain embodiments, administration of the subject composition results in an extended therapeutic effect of a magnitude that is not possible with other modes of administration of the analgesic agent. In certain embodiments, such administration results in therapeutically effective relief of pain or other disease or condition for a prolonged period, such as 12 hours, 18 hours, a day, three days, or even a week or more. Such extended therapeutic effect may be characterized by in vivo experimentation. For example, administration of a therapeutically effective amount of the composition to a rat may result in doubling of a paw withdrawal latency time in a hot plate test for at least about 12 hours, 18 hours, or 1, 2, 3, 4, 5, 6, 7 or more days.

In another aspect, the present invention is directed to methods of using the subject compositions for prophylactic or therapeutic treatment. In certain instances, the subject compositions may be used to prevent or relieve pain in a patient. In certain embodiments, use of the subject compositions, which release in an extended therapeutic effect as compare to other modes of administration, allow for different treatment regimens than are possible with such other modes of administration.

In another aspect, the subject compositions may be used in the manufacture of a medicament for any number of uses, including for example treating any disease or other treatable condition of a patient. In other embodiments, this invention contemplates a kit including subject compositions, and optionally instructions for their use and optionally a device for administration, such as a syringe. Uses for such kits include, for example, therapeutic applications.

These embodiments of the present invention, other embodiments, and their features and characteristics will be apparent from the description, drawings, and claims that follow.

### BRIEF DESCRIPTION OF THE DRAWINGS

FIG. 1 depicts plasma levels of lidocaine after administration of the subject composition as described in Example 3.

FIG. 2 depicts injection site as described in Example 4.

FIG. 3 depicts the results of experiments described in Example 5 relating to in vivo release of lidocaine HCl in sesame oil.

FIG. 4 depicts the results of a Randall-Selitto test described in Example 6.

### DETAILED DESCRIPTION OF THE INVENTION

1. Overview

The present invention relates in part to pharmaceutical compositions comprising pharmaceutically acceptable salts of an analgesic substance and an oil. Other embodiments relate to methods of making and using such pharmaceutical compositions. The present invention also relates to methods of administering such pharmaceutical compositions, e.g., as part of a treatment regimen, for example, subcutaneously or intramuscularly.

In certain aspects, the subject pharmaceutical compositions, upon contact with body fluids including blood, spinal fluid, lymph or the like, exhibit a therapeutic effect over a sustained and extended period (as compared to the therapeutic effect obtained from administration of the same analgesic salt in an isotonic saline solution or other modes and methods of administration). Such a system may result in a prolonged therapeutic effect (over, for example, 8 to 800 hours, preferably 24 to 480 or more hours) using effective amounts (e.g., 0.0001 mg/kg/hour to 100 mg/kg/hour) of the analgesic agent. This dosage form may be administered as is necessary depending on the subject being treated, the severity of the affliction, the judgment of the prescribing physician, and the like.

By way of example and without limitation, morphine sulfate and other injectable opioids are widely used epidurally in post-operative settings, while lidocaine HCl and other caine analgesic salts have been used locally, for the control of pain. Typically, these analgesic salts have a polar character and are dissolved in a polar solvent, e.g. water or saline, for administration. Local regions where these analgesic salts may be used include surgical resection sites, open wounds or any otherwise afflicted areas, such as cavities. For example, the need for this type of administration may arise in the treatment of incisional wounds following surgery, post-operative and postpartum pain, as well as more serious traumas such as wounds caused by accidents or recesses or cavities caused by the removal of tumors from bones. Although the analgesic is effective in reducing the pain, the therapeutic effect typically will last only a few of hours when administered in such fashion, e.g., in a saline bolus. For administrations to be more effective, the therapeutic effect of the agent once administered must be prolonged over a period of time. As taught by the present invention, this result has been achieved by incorporating a pharmaceutically acceptable salt form of an analgesic into an oil such that the salt is sparingly soluble, slightly insoluble, very slightly insoluble, or practically insoluble in the resulting pharmaceutical composition.

It has been observed that the therapeutic effect of an analgesic may be achieved over an extended period time upon administration to a subject of the inventive pharmaceutical compositions as compared to other modes of administration. Without limiting the invention to a particular mechanism of action or otherwise circumscribing the scope of the teachings herein, it is possible that the extended effect is caused by sustained release of the subject analgesic from the oil. This slow release may be attributable to a reduced solubility of the pharmaceutically acceptable salt of the analgesic agent in the oil, which may slow the process by which the agent comes into contact with the body fluids of the subject. Alternatively, the oil may act as a coating on the analgesic agent through which the agent must diffuse before coming directly into contact with body fluids. It is not known whether the subject analgesic is released from the subject compositions as the salt or the neutral form thereof, an outcome which may depend on the pKa of the subject analgesic and the micro-pH of the local environment. In any case, it is believed that the salt of the analgesic agent does not dissolve in the oil but instead remains as a fine crystalline or other solid particle in the oil. The nature of the oil and analgesic salt mixture, whether best characterized as an emulsion, a suspension or the like, has not yet been determined. It may also be the case that other mechanisms are responsible for the extended therapeutic effect observed for the subject compositions.

2. Definitions:

When used with respect to an analgesic agent or other material, the term “sustained release” or “controlled release” is art-recognized. For example, a subject composition which releases a substance over time may exhibit sustained release characteristics, in contrast to a bolus type administration in which the entire amount of the substance is made biologically available at one time. As described above, it may be the case that the extended therapeutic effect observed for the inventive compositions is a result of sustained release. Such a sustained release, if occurring, may result in prolonged delivery of therapeutically effective amounts of any incorporated therapeutic agent.

The term “local anesthetic” is art-recognized and includes drugs which provide local numbness or pain relief.

The terms “analgesic”, “analgesic agent”, “analgesic substance” and the like are art-recognized and includes therapeutic agents which treat pain and other physical sensations. The terms, and a variety of such agents, are described in more detail below. The term “salt of an analgesic agent”, “analgesic salt” and the like refers to a pharmaceutically acceptable salt of an analgesic agent, as further described below.

The term “oil” is art-recognized, and includes any material that has non polar and hydrophobic properties, such that polar substances, such as certain pharmaceutically acceptable salts of analgesic agents, are sparingly soluble, slightly soluble, very slightly soluble, practically insoluble in an oil. This term is discussed in more detail below.

The terms “biocompatible oil” and “biocompatibility” when used in relation to oils are art-recognized. For example, biocompatible oils include oils that are neither themselves toxic to the subject, nor degrade (if the oil degrades) at a rate that produces byproducts at toxic concentrations in the subject. Consequently, in certain embodiments, toxicology of a oil intended for in vivo use may be determined after one or more toxicity analyses which are known in the art. It is not necessary that a biocompatible oil used in a subject composition have a purity of 100% for the composition or the oil to be deemed biocompatible; indeed, it is only necessary that the subject compositions be biocompatible as set forth above.

The phrases “parenteral administration” and “administered parenterally” are art-recognized terms, and include modes of administration other than enteral and topical administration, such as injections, and include, without limitation, intravenous, intracarpal, transcardiac, parasternal, intramuscular, intraarterial, intrathecal, intracapsular, intraorbital, intracardiac, intradermal, intraperitoneal, intraventricular, transtracheal, subcutaneous, subcuticular, intra-articular, subcapsular, subarachnoid, intraspinal, epidural and intrasternal injection and infusion.

The phrase “internal administration” includes any mode of administration other than topical, i.e., application to the skin of a subject.

The term “treating” is an art-recognized term which includes curing as well as ameliorating at least one symptom of any condition or disease, and includes prophylactic treatments.

A “patient,” “subject,” or “host” to be treated by the subject method may mean either a human or non-human animal, such as primates, mammals, and vertebrates.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing”, when used in relation to pain or another medical condition, is well understood in the art. The term includes, for example, administration of a composition containing an analgesic which reduces the intensity of, or delays the onset of, sensations of pain in a subject relative to a subject which does not receive the composition.

The phrases “systemic administration,” “administered systemically,” “peripheral administration” and “administered peripherally” are art-recognized, and include the administration of a subject composition at a site remote from the disease being treated. Administration of an agent directly into, onto or in the vicinity of a lesion of the disease being treated, even if the agent is subsequently distributed systemically, may be termed “local” or “regional” administration, other than directly into the central nervous system, e.g., by subcutaneous administration, such that it enters the patient's system and, thus, is subject to metabolism and other like processes.

The phrase “therapeutically effective amount” is an art-recognized term. In certain embodiments, the term refers to an amount of the salt of an analgesic agent that, when incorporated into an oil of the present invention, produces some desired effect at a reasonable benefit/risk ratio applicable to any medical treatment. In certain embodiments, when the analgesic substance is an analgesic, for example, the term refers to that amount necessary or sufficient to eliminate or reduce sensations of pain for a period of time. For certain subject pharmaceutical compositions, that period of time is greater than the period of time achieved by other compositions containing the same analgesic (as a salt or otherwise) or other modes of administration. In other embodiments, a therapeutically effective amount of a pharmaceutical composition having an analgesic salt, such as lidocaine HCl or an analog thereof, and an oil for in vivo use in a subject will likely depend on a number of factors, including the chemical and physical characteristics of the oil, the identity of the oil, the solubility of the salt in the oil, the amount of analgesic salt incorporated in the oil, and the method of administration. Also, the effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

The terms “incorporated” and “encapsulated” are art-recognized when used in reference to an analgesic agent or other material and an oil. In certain embodiments, these terms include incorporating, formulating or otherwise including such agent into a subject composition which also contains an oil. The terms contemplate any manner by which an agent or other material is mixed or combined with an oil. The term “co-incorporation” or “co-encapsulation” refers to the incorporation of an analgesic agent or other material and at least one other therapeutic agent or other material in a subject composition.

The terms “granulated”, “mixed”, “wetted”, “dried”, “milled”, “pulverized” and “blended” are art-recognized when used in reference to a therapeutic agent or other material and an oil, such as a composition of the present invention.

The term “sieve” and “sieved” are art-recognized when used in reference to size classification to obtain a desired particle size.

The term “therapeutic agent” is art-recognized and includes an agent capable of having a desired therapeutic effect on a subject. Analgesic agents are one example of therapeutic agents. Certain therapeutic agents are capable of preventing the establishment or growth (systemic or local) of a tumor or infection. Examples include boron-containing compounds (e.g., carborane), chemotherapeutic nucleotides, drugs (e.g., antibiotics, antivirals, antifingals), enediynes (e.g., calicheamicins, esperamicins, dynemicin, neocarzinostatin chromophore, and kedarcidin chromophore), heavy metal complexes (e.g., cis-platin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals. Another therapeutic agent is an “augmenting agent”, which is described in detail below.

“Small molecule” is an art-recognized term. In certain embodiments, this term refers to a molecule which has a molecular weight of less than about 2000 amu, or less than about 1000 amu, and even less than about 500 amu. Certain small molecules are therapeutic agents.

The term “solubility” is art-recognized. In certain embodiments, the solubility is expressed in terms of an amount of solvent required to dissolve an amount of solute, or matter, at a specified temperature. In certain embodiments, the solubility of a substance which is less than 0.01 mol/L is insoluble, solubility of a substance greater than 0.1 mol/L soluble, and between 0.01 and 0.1 mol/L slightly soluble. In another certain embodiment, solubility is expressed in subjective terms. (For further discussion, see Remington's Pharmaceutical Sciences (Ed. by AR Gennaro) 19^{th} ed. 1995.) For illustration purposes, solubility terms and properties are presented below:

| Descriptive term | Parts of solvent needed for 1 part solute |
|---|---|
| Very soluble | <1 |
| Freely soluble | 1-10 |
| Soluble | 10-30 |
| Sparingly soluble | 30-100 |
| Slightly soluble | 100-1000 |
| Very slightly soluble | 1000-10,000 |
| Practically insoluble | >10,000 |

The term “solution” is art-recognized. In certain embodiments, solution means a mixture of one compound mixed with another. The terms “solvent” and “solute” are art-recognized. The compound that is present in the larger amount in a solution is called the solvent and the other part is called the solute. For purposes of this inventions the analgesic agent will be known as the solute and the oil as the solvent, event if the amount of analgesic exceeds the amount of oil. By use of the terms solvent and solute to describe the present invention, it is not intended to indicate that, for any particular embodiment, an analgesic salt is fully soluble in an oil, but only that the two components are mixed together.

A “polar solvent” is an art-recognized term. A polar solvent contains substances with asymmetric charge distribution. A “non-polar” solvent is an art-recognized term. In general, a non-polar solvent will dissolve non-polar molecules, and a polar solvent will dissolve polar molecules. Semi-polar solvents may induce a degree of polarity in non-polar molecules. The solubility of a substance in a given solvent is largely a function of the polarity of a solvent as compared to the polarity of the substance.

The “dielectric constant” (∈) of a compound, such a solvent, is an art-recognized term. The dielectric constant, ∈ is an index of its polarity. A series of solvents of increasing polarity will show a similar increase in dielectric constant. Solvents may be generally classified according to their dielectric constants as polar (∈>50), semi-polar (∈=20-50), or non-polar (∈=1-20), measured at 20° C. The oils of the present invention are generally non-polar.

“Hydrophobic” and “hydrophilic” are art-recognized terms. In general, hydrophobic refers to non-polar substances, and hydrophilic refers to polar substances. A hydrophobic solvent is generally non-polar. A hydrophilic solvent is generally polar.

The term “fluid” is art-recognized to refer to a non-solid state of matter in which the atoms or molecules are free to move in relation to each other, as in a gas or liquid. If unconstrained upon application, a fluid material may flow to assume the shape of the space available to it. A fluid material may also be termed “flowable.” This term is art-recognized and includes, for example, pharmaceutical compositions that are capable of being sprayed into a site; injected with a manually operated syringe fitted with, for example, a 23- or 18-gauge needle; or delivered through a catheter. Also included in the term “flowable” are those highly viscous, “gel-like” materials at room temperature that may be delivered to the desired site by pouring, squeezing from a tube, or being injected with any one of the commercially available injection devices that provide injection pressures sufficient to propel highly viscous materials through a delivery system such as a needle or a catheter. In certain instances, flowable subject compositions have the ability to assume, over time, the shape of the space containing it at body temperature.

Viscosity is an art recognized term, wherein viscosity is the resistance of a fluid to flow. Absolute viscosity of a liquid is an art-recognized term, wherein the viscosity is measured in units of (mass)/(cm)(sec). Kinematic viscosity is an art-recognized term where it is defined as the ratio of the absolute viscosity to the density of a liquid. A unit of kinematic viscosity is the stoke, expressed in square centimeters per second. The customary unit of kinematic viscosity is the centistoke, cSt, which is one one-hundredth of a stoke. In one embodiment, viscosity is measured by a rheometer. In another embodiment, viscosity is measured by the force of gravity to produce flow through a capillary tube at a controlled temperature.

The phrase “pharmaceutically acceptable” is art-recognized. In certain embodiments, the term includes compositions, analgesic agents, other therapeutic agents, oils and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” is art-recognized, and includes relatively non-toxic, inorganic and organic acid addition salts of molecules, including without limitation, analgesic agents, other therapeutic agents, and other materials. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. For more examples of suitable salts, see, for example, J. Pharm. Sci., 66:1-19 (1977). With respect to pharmaceutically acceptable salts of analgesic agents for use in the subject compositions, further information on the characteristics are set forth below.

The phrase “pharmaceutically acceptable carrier” is art-recognized, and includes, for example, pharmaceutically acceptable materials, compositions or vehicles, such as a liquid or solid filler, diluent, excipient or solvent, involved in carrying or transporting any pharmaceutical composition from one organ, or portion of the body, to another organ, or portion of the body. Each carrier must be “acceptable” in the sense of being compatible with the other ingredients of the pharmaceutical composition and not injurious to the subject. In certain embodiments, a pharmaceutically acceptable carrier is non-pyrogenic.

The term “drug delivery device” is an art-recognized term and refers to any medical device suitable for the application of the subject pharmaceutical compositions to a targeted organ or anatomic region.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Moreover, the term “alkyl” (or “lower alkyl”) includes both “unsubstituted alkyls” and “substituted alkyls”, the latter of which refers to alkyl moieties having substituents replacing a hydrogen on one or more carbons of the hydrocarbon backbone. Such substituents may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN, and the like.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means —SH; the term “hydroxyl” means —OH; and the term “sulfonyl” means —SO₂⁻.

The terms “amine” and “amino” are art-recognized and include both unsubstituted and substituted amines, e.g., a moiety that may be represented by the general formulas:

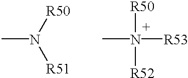

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

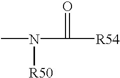

wherein R50 is as defined above, and R54 represents a hydrogen, an alkyl, an alkenyl or —(CH₂)ₘ—R61, where m and R61 are as defined above.

The term “amido” is art-recognized as an amino-substituted carbonyl and includes a moiety that may be represented by the general formula:

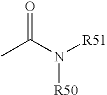

wherein R50 and R51 are as defined above. Certain embodiments of the amide in the present invention will not include imides which may be unstable.

The term “alkylthio” is art-recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art-recognized and includes such moieties as may be represented by the general formulas:

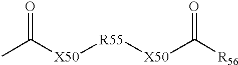

wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art-recognized and includes a moiety that may be represented by the general formula:

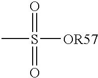

in which R57 is an electron pair, hydrogen, alkyl, cycloalkyl, or aryl.

The term “sulfate” is art-recognized and includes a moiety that may be represented by the general formula:

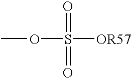

in which R57 is as defined above.

The term “sulfonamido” is art-recognized and includes a moiety that may be represented by the general formula:

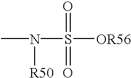

in which R50 and R56 are as defined above.

The term “sulfamoyl” is art-recognized and includes a moiety that may be represented by the general formula:

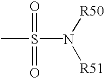

in which R50 and R51 are as defined above.

The term “sulfonyl” is art-recognized and includes a moiety that may be represented by the general formula:

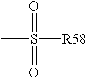

in which R58 is one of the following: hydrogen, alkyl, alkenyl, alkynyl, cycloalkyl, heterocyclyl, aryl or heteroaryl.

The term “sulfoxido” is art-recognized and includes a moiety that may be represented by the general formula:

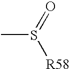

in which R58 is defined above.

The term “phosphoramidite” is art-recognized and includes moieties represented by the general formulas:

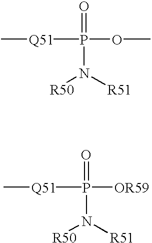

wherein Q51, R50, R51 and R59 are as defined above.

The term “phosphonamidite” is art-recognized and includes moieties represented by the general formulas:

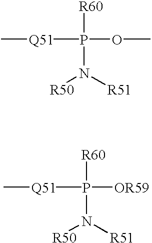

wherein Q51, R50, R51 and R59 are as defmed above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

The term “selenoalkyl” is art-recognized and includes an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art-recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain aspects of the composition of the present invention may exist in particular geometric or stereoisomeric forms, including the analgesic agents or other therapeutic agents. In addition, compounds included in compositions of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. The term “hydrocarbon” is art recognized and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” is art-recognized and includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, New York, (1991).

The phrase “hydroxyl-protecting group” is art-recognized and includes those groups intended to protect a hydroxyl group against undesirable reactions during synthetic procedures and includes, for example, benzyl or other suitable esters or ethers groups known in the art.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59, McGraw Hill Book Company, New York, (1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

Contemplated equivalents of the analgesic agents, other therapeutic agents, oils and other materials and compositions described above include such materials which otherwise correspond thereto, and which have the same general properties thereof (e.g., biocompatible, analgesic, non-polar, etc.), wherein one or more simple variations of substituents or other entities are made which do not adversely affect the efficacy of such molecule to achieve its intended purpose. In general, the compositions and components of the present invention may be prepared by the methods illustrated in the general schemes as, for example, are described below, or by modifications thereof, using readily available starting materials, reagents and conventional procedures. In these reactions, it is also possible to make use of variants which are in themselves known, but are not mentioned here.

3. Exemplary Subject Compositions, and Methods of Making and Using the Same

A. Analgesic Salts

Pharmaceutically acceptable salts of analgesic agents may be used in the present invention and include biologically, physiologically, or pharmacologically active substances that act locally or systematically in a subject to treat pain or other physical sensations (among other things) in a subject. Examples of suitable pharmaceutically acceptable salts of analgesics are set forth below. Other analgesic salts which may be used in the present invention are known to those of skill in the art.

A variety of different analgesics are known in the art, including opiate agonist or antagonists and synthetic piperdine analgesics as well as local analgesics. Suitable analgesic salts include the salt of an opiate agonist or antagonist, or a salt of a synthetic piperdine analgesic, or salts of non-opioid pain receptor agonists, such as salts of analgesic non steroidal anti-inflammatory drugs.

Other suitable analgesic salts include salts of opiate agonists and antagonists such as: anileridine phosphate, anileridine dihydrochloride, buprenorphine HCl, butorphanol tartrate, codeine acetate, hydromorphone HCl, levorphanol HBr, levorphanol HCl, levomethadyl acetate HCl, meperidine HCl, morphine sulfate, nalbuphine HCl, oxycodone HCl, oxymorphone HCl, pentazocine HCl, propoxyphene HCl, fentanyl citrate, butorphanol, alfentanil HCl, sufentanyl citrate, remifentanyl citrate, terfentanyl citrate, and naltrexone HCl.

Salts of peptides and peptidomimetics that bind to one or more neuroreceptors such as the δ-opioid, μ-opioid, κ-opioid, and ε-opioid are considered analgesic salts and may be used in the present invention. Such compounds include salts of enkephalins, endorphins, casomorphins, and kyotorphins.

Other suitable analgesic agents include salts of non-opioid agonist analgesics. Salts of non-opioid agonists include salts of α-2 adrenergic receptor agonists, such as clonidine HCl, tizanide HCl and medetomidine HCl. Other salts of non-opioid agonists include salts of N-methyl-D-aspartate receptor antagonists such as ketamine HCl and dextromethrophan HBr. Other analgesic salts that act on non-opioid pain receptors include salts of somatostatin analogs such as sandostatin octreotide acetate, and salts of other non-opioid pain receptor agonists and antagonists.

A subject composition may also comprise salts of analgesic non steroidal anti-inflammatory drugs, such as ketorolac tromethamine, diclofenac sodium, fenoprofen calcium, ibuprofen sodium, ketoprofen sodium, meclofenamate sodium, naproxen sodium, tolmentin sodium.

A subject composition may also comprise analgesic agents which are highly polar, and thus exhibit the same solubility characteristics as salts of analgesic agents in oils of the subject invention. Such highly polar agents are also deemed analgesic salts for all purposes of this invention unless otherwise expressly provided herein.

Certain analgesics are often used to treat pain. The structures of representative analgesic salts, e.g., lidocaine HCl, dibucaine HCl, bupivacaine HCl, etidocaine HCl, mepivacaine HCl, prilocaine HCl, benzocaine HCl, butanilicaine HCl, trimecaine HCl, chloroprocaine HCl, procaine HCl, propoxycaine HCl, tocainide HCl, and tetracaine HCl are presented below.

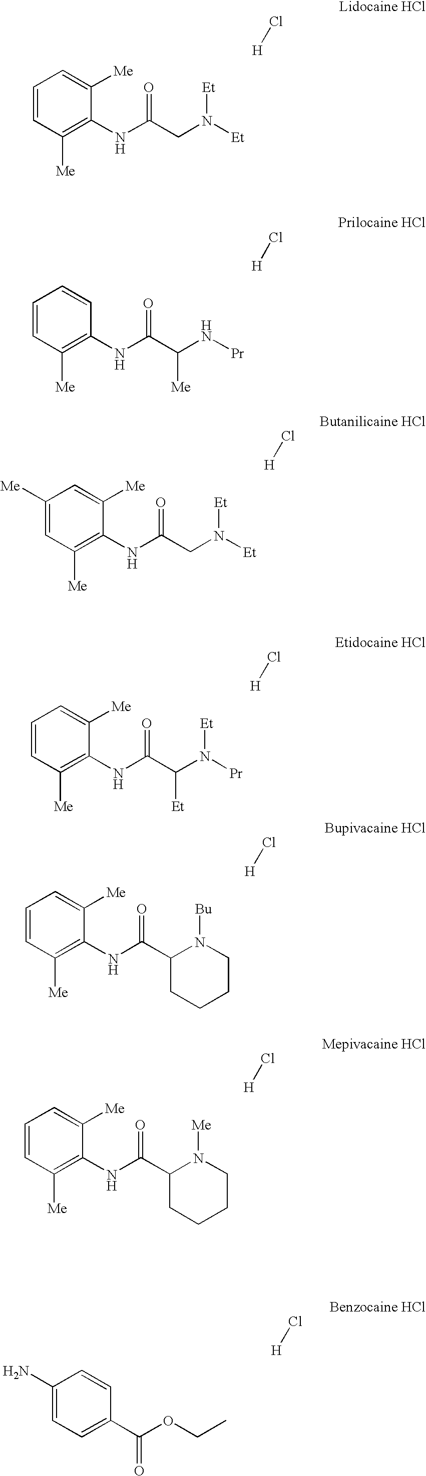

The above salts of local analgesic agents thus represent a family of related compounds, referred to herein as salts of “caine analgesics”, which caine analgesics have in common (i) a core comprising an aryl ring directly bound to an amide or ester group, and (ii) an amino group, which may represent a primary, secondary, or tertiary amine, and may be linked to either the aryl or amide/ester portion of the core. In certain embodiments, a caine analgesic has an aryl core linked to a secondary or tertiary amine through an ester or amide linkage.

A variety of other suitable salts of analgesics are known in the art and such analgesic salts may be employed in the subject compositions and methods without departing from the spirit or scope of the present invention.

In certain embodiments, the particle size of the salt of an analgesic may be varied. For example, article size distribution may be a function of the total grinding time, with shorter grinding times producing, on average, larger particles, and longer grinding times producing, on average, smaller particles. The size range of a sample of microparticles produced in this way can be further restricted by sieving to obtain particle sizes of about less than 150 μm, 100 μm, 75 μm, 50 μm, 25 μm, or even less than 10 μm.

B. Oils

A variety of oils may be used in the subject invention. An oil may be of natural or synthetic origin and may contain fatty acids or lipids of different lengths within their structure. An oil which may be used in the subject invention is any oil acceptable for pharmaceutical applications. Mixtures of oils are included in the term “oil” as defined herein. Oils may have impurities, especially those derived from natural sources.

An oil may be composed of any neutral or non-polar lipid, including acylglycerols, fatty acids, hydrocarbons, terpenes, phenolic lipids, lipid quinones, sterols and the like, all optionally substituted, and mixtures thereof. Alternatively, oils may be catalogued by source, such as vegetable oils, animal oils, mineral oils, etc.

Acylglycerols include monoglycerides having one fatty acid esterified to glycerol, diglycerides having two fatty acids esterified to glycerol, and triglycerides having three fatty acids esterifying the three alcohol groups of glycerol.

Fatty acids include esterfied, optionally substituted, long carbon chains with variations in the branching of, number of double bonds in, and length of the chains. Fatty acids also include saturated fatty acids, monoenoic and polyenoic fatty acids, mono- and multibranched fatty acids and ring containing fatty acids. In a particular embodiment, a fatty acid has more than 8, 12, or 24 carbon atoms.

Hydrocarbons oils include paraffins. Terpenes include squalene and carotenoid compounds. Phenolic lipids are formed from a catechol, a resorcinol or a hydroquinone group, substituted or not, linked to a saturated or unsaturated carbon chain. Lipid quinones have one or two hetero- or carbocycles with isoprenoid side chains of variable length and number of double bonds. They are either vitamins, for example, vitamin K, or coenzymes, such as coenzyme Q or ubiquinones and plastoquinones. Sterols include, for example, cholesterol.

Vegetable oils, derived from plants and seeds, are known in the art to be a broad class of triglycerides, and include hydrogenated vegetable oils, partially hydrogenated vegetable oils, refined vegetable oils, synthetic triglycerides, modified triglycerides, fractionated triglycerides, and mixtures thereof. Exemplary vegetable oils include soy bean oil, palm oil, rapeseed oil, sunflower oil, corn oil, olive oil, sesame oil, grape seed oil, poppy seed oil, linseed oil, almond oil, walnut oil, cacao oil, kukui oil, canola oil, castor oil, coconut oil, cottonseed oil, peanut oil, safflower oil, and wheat germ oil.

Sesame oil, for example, is an oil derived from the seeds of varieties of Sesamum indicum L., Pedaliaceae. Sesame oil may consist of olein, stearin, palmitin, myristin, linolein, sesamin, sesamolin, and other compounds. Sesame oil includes glycerides of the fatty acids linoleic, oleic, palmitic and stearic, and may include other substances.

Other suitable oils include oil products derived from animal sources, such as fish oil. Other oils are mineral oil products.

A variety of other suitable oils are known in the art, and such other oils may be employed in the subject compositions and methods without departing from the spirit or scope of the present invention.

In certain embodiments, an oil has a low dielectric constant, e.g. ε less than about 50, 20, 10 or 5 at 20° C.

An oil may be biodegradable or non-biodegradable. Examples of potentially biodegradable oils include oils derived from plants and animals, vegetable oils, paraffin oils, or triglyceride derivatives such as MIGLYOL® or mixtures thereof.

A oil may be liquid at room temperature, and a solid or more viscous oil may be heated to form a liquid prior to administration. In certain embodiments, the oil is flowable at room temperature or alternatively at least the body temperature of the subject. Expressed as a viscosity, in certain embodiments the oil has a viscosity that is below about 140 cSt or 90 cST, and in the same or other embodiments, the oil has a viscosity that is above about 20 cSt. In certain embodiments, the oil has a viscosity that is from about 60-90 cSt, or about 70 cSt.

C. Pharmaceutical Compositions

Certain pharmaceutical compositions of the present invention include (i) a pharmaceutically acceptable salt of an analgesic agent, and (ii) an oil. A representative method of making the subject compositions is set forth in the Examples and other suitable methods are known to one of skill in the art.

In certain embodiments of the present invention, different pharmaceutically acceptable salt of an analgesic agent may be combined with different oils to provide a pharmaceutical composition that achieves as therapeutic effect over an extended period of time upon administration to a subject. For example, the different salts and oils may be combined and tested empirically to determine which combinations achieve the desired results without undue experimentation.

It is possible to distinguish the length of extended therapeutic effect achieved by the subject compositions by comparison to other formulations and modes of administration of the same analgesic salt. For example, certain subject embodiments may exhibit a therapeutic effect for a period of time that is at least about 25%, 50%, 75% or 100% longer, or even at least about two, three, four, five or even more times as long, as the therapeutic effect observed for administration of the same salt by the same or similar means in saline, water or other pharmaceutically acceptable solvent in which the salt is at least soluble.

Alternatively, a salt and an oil may be chosen so that the salt be sparingly soluble, slightly soluble, very slightly soluble or practically insoluble in the oil or the resulting pharmaceutical composition (if the pharmaceutical composition has components in addition to the salt and the oil). For example, the solubility of lidocaine HCl in sesame oil is about 9.23×10⁻⁵ mol/L at 25° C. In other embodiments, the analgesic salt does not appreciably dissolve in the pharmaceutical composition upon combination of the salt, the oil and optionally other materials. This case may be distinguished from the case in which the analgesic salt is first dissolved in a polar solvent in which it is at least soluble, usually highly soluble, and then the salt and solvent mixture is added to the oil to form the pharmaceutical composition. In such a case, the salt is best understood to dissolve or be soluble in the pharmaceutical composition because the salt is first dissolved in a polar solvent before being combined with a non-polar substance. It may be the case that had the same amounts of polar solvent, analgesic salt and oil been combined at one time, the salt would not be appreciably dissolved or at most sparingly soluble in the resulting pharmaceutical composition.

In certain embodiments, the subject compositions comprise about 1% to about 90%, alternatively about 2% to about 50%, by weight of a pharmaceutically acceptable salt of analgesic agent in a pharmaceutical composition. In certain embodiments, a composition comprises at least about 1% by weight of a salt of an analgesic agent, more particularly at least about 2%, at least about 4%, at least about 10%, at least about 20%, at least about 50%, or even more than about 90% of said salt.

In certain embodiments, the subject compositions comprise a pharmaceutically acceptable salt of an analgesic agent and at least about 33%, 50%, 66% 75%, 80%, 90%, 95% or more by weight of one or more oils. In certain embodiments, the pharmaceutical composition comprises pharmaceutically acceptable salts of one or more analgesic agents and one or more oils, without more. In such embodiments, the amount by weight of such oils is 100% less the weight percentage of such salts.

In certain embodiments, the subject pharmaceutical composition may contain materials other than the pharmaceutically acceptable salt of an analgesic agent that may be hydrophobic or hydrophilic. In certain embodiments, the pharmaceutical composition does not contain any appreciable amount of a hydrophilic component (whether as part of the oil or otherwise). It is understood that oils used in the present invention may have minor amounts, even trace amounts, of hydrophilic materials, which materials do not affect the bulk physical characteristics of the oil, such as its non-polar character. In other embodiments, the pharmaceutical composition has less than 25%, 20%, 15%, 10%, 5%, or even less than 1% of a hydrophilic component. For those subject compositions containing such other materials (other than small amounts in the oil), it may be important in certain of such embodiments to maintain the polarity of the resulting pharmaceutical composition so that the pharmaceutically active salt of an analgesic agent contained therein is at most only sparingly soluble in the resulting pharmaceutical composition.

The subject pharmaceutical compositions may also include therapeutic agents in addition to the analgesic agent. For example, the other therapeutic agent may be the neutral form of the analgesic salt in the composition. In this way, the magnitude, duration and other features of the therapeutic effect achieved upon administration of the subject compositions to a subject may be varied. Alternatively, such other therapeutic agents may be administered along with the subject composition but formulated differently.

By way of another example, an analgesic formulation of the present invention may include an “augmenting agent” or “augmenting compound”, and certain of the augmenting agents may be analgesics hereunder, as will be known to one of skill in the art. One class of augmenting agent are the glucocorticosteroids, such as dexamethasone, cortisone, prednisone, hydrocortisone, beclomethasone dipropionate, betamethasone, flunisolide, methylprednisone, paramethasone, prednisolone, triamcinolone, alclometasone, amcinonide, clobetasol, fludrocortisone, diflorasone diacetate, fluocinolone acetonide, fluocinonide, fluorometholone, flurandrenolide, halcinonide, medrysone and mometasone and pharmaceutically acceptable mixtures thereof and salts thereof or any other suitable art-known glucocorticosteroid, either naturally occurring or synthetic.

Examples of non-glucocorticosteroid augmenting compounds which may also be effective when co-administered with an analgesic include alkalinizing agents, non-glucocorticoid steroids such as neuroactive steroids, modulators of gamma amino butyric acid receptors, modulators of ionic transport across cell membranes, antipyretic agents, adrenergic receptor agonists or antagonists, tubulin binding agents, osmotic polysaccharides, agonists and antagonists of potassium ATP channels, Na, K-ATPase inhibitors and enhancers, neurokinin antagonists, phosphatidylinositol-specific phospholipase C (“PLC”) inhibitors, inhibitors of leukocyte glucose metabolism, anti-convulsants, analeptics, tranquilizing agents, antidepressants, convulsants, leukotrienes and prostaglandin agonists and inhibitors, phosphodiesterase agonists and inhibitors, vasoconstrictive agents in sustained release form, and combinations of any of the foregoing.

These compounds, both glucocorticoids and non-glucocorticoids, may increase the effectiveness of the analgesic, and may additionally reduce inflammation or other unwanted symptoms related to the pain.

In one embodiment, the augmenting agent includes an alkalinizing agent. The alkalinizing augmenting agents used herein preferably raise the pH of the medium in which the analgesic agents in sustained release form are present (e.g., either an injection medium or the environment at the site of injection) to provide a pH from about 6.0 to about 8.5, preferably from about 7.5 to about 8.5. Preferably, the alkalinizing agent may be, for example, a carbonate buffer such as sodium carbonate. Of course, any other alkalinizing agent that is pharmaceutically acceptable may also be effectively employed.

The augmenting agents also include non-glucocorticosteroids, e.g., androgens, such as testosterone and its active derivatives, analogs, and metabolites; estrogens, such as estradiol and its active derivatives, analogs, and metabolites and progestins, such as progesterone and its active derivatives, analogs, and metabolites, and mixtures of any of these.

In another embodiment, the augmenting agent is a neuroactive steroid, such as, e.g., one or more of the class of anesthetic steroids. Neuroactive steroids useful as augmenting agents according to the invention also include those which modulate GABA receptors. Suitable neuroactive steroids include, simply by way of example, althesin and its main component, alphaxalone and active analogs, derivatives and mixtures thereof, as well as 5-alpha-pregnane-3 alpha-21-diol-20-one (tetrahydro-deoxycorticosterone or THDOC) and/or allotetrahydrocortisone (the 17-beta configuration); and dehydroepiandrosterone (“DHE”) and active analogs, derivatives and mixtures thereof. In certain embodiments, the neuroactive steroids are present as an additive in the subject pharmaceutical compositions in a concentration ranging from about 0.01 to about 1,2 or 3% by weight, and most preferably from about 0.05 to about 0.5% by weight.

Suitable augmenting agents also include non-steroidal modulators of GABA receptors, including those that are capable of potentiating the inhibitory effects of GABA on those receptors. Such compounds include the benzodiapenes, e.g., diazepam as well as its active derivatives, analogs, and metabolites, and mixtures thereof. In certain embodiments, the diazepam is present as an additive in the vehicle in a concentration ranging from about 0.01 to about 1,2 or 3% by weight, or from about 0.05 to about 0.5% by weight. Of course, the artisan will appreciate that the potency of benzodiazapenes varies widely, as do all augmenting agents, and will adjust these concentration ranges accordingly for other benzodiazapenes, relative to the potency of diazepam.

In yet another aspect of the invention, the augmenting agent is a modulator of ionic transport across cell membranes. Monovalent and multivalent metal ion transport may be modulated. Agents include, e.g., sodium, potassium and calcium channel modulators (e.g., nifedipine, nitrendipine, verapamil, etc.). In certain embodiments, these also include, but are not limited to, aminopyridine, benzamil, diazoxide, 5,5-diphenylhydantoin, minoxidil, tetrethylammonium and valproic acid. In certain embodiments, the ion transport modulating agent is present as an additive in the composition in a concentration ranging from about 0.01 to about 5, 10 or 15% by weight, or from about 0.05 to about 1.5% by weight.

Augmenting agents also include, e.g., antipyretic agents such as aminopyrine, phenazone, dipyrone, apazone, phenylbutazone and derivatives and analogs thereof. Aminopyrine may be included in the composition in a concentration ranging from about 0.01 to about 0.5, 1.0 or 1.5%, or from about 0.05 to about 0.5%, by weight.

Other suitable augmenting agents include, e.g., adrenergic receptor modulators, such as α2 receptor agonists, can also be used as augmenting agents. Simply by way of example, the α2 receptor agonist clonidine provides useful augmentation of local anesthesia, although any other art known α2 receptor modulators capable of augmenting local anesthesia according to the invention may be used. Clonidine may be included in the composition in a concentration ranging from about 0.01 to about 0.5, 1.0, or 1.5%, or from about 0.05 to about 1.0%, by weight.

Tubulin binding agents that are capable of promoting the formation or disruption of cytoplasmic microtubules are may be employed as augmenting agents according to the invention. Such agents include, for example, colchicine and the vinca alkaloids (vincristine and vinblastine) as well as active derivatives, analogs metabolites and mixtures thereof. Of course, some agents may be classified in more than one category, as, for example, colchicine is also known to inhibit glucose metabolism in leukocytes. Colchicine may be included in the composition in a concentration ranging from about 0.01 to about 1.0, 1.5 or 3%, or from about 0.05 to about 0.5%, by weight.

Other embodiments of the invention provide potassium-ATP channel agonists for use as augmenting agents. A suitable potassium-ATP channel agonist is, for example, diazoxide, as well as its active derivatives, analogs, metabolites and mixtures thereof are useful as augmenting agents.

Sodium/potassium ATPase inhibitors are also useful as augmenting agents according to the invention. In certain embodiments, the sodium/potassium ATPase inhibitors are cardiac glycosides that are effective to augment local anesthesia. Cardiac glycosides that are useful according to the invention include, e.g., oubaine, digoxin, digitoxin and active derivatives, analogs, and metabolites, and mixtures of any of these.

Additionally, augmenting agents according to the invention include, e.g., neurokinin antagonists, such as, e.g., spantide and other peptide inhibitors of substance P receptors that are well known to the art, e.g., as are listed in Receptor and Ion Channel Nomenclature Supplement, Trends in Pharmacological Sciences 18:64-65. PLC inhibitors and anti-seizure agents and agents that stabilize cell membrane potential, such as, e.g., benzodiazepines, barbiturates, deoxybarbiturates, carbamazepine, succinamides, valproic acid, oxazalidienbiones, phenacemide and active derivatives, analogs and metabolites and mixtures thereof. In certain embodiments, the anti-seizure augmenting agent is phenytoin, and most preferably is 5,5-diphenylhydantoin.

Locally acting vasoconstrictive agents also provide effective augmentation of local anesthesia superior to that provided by immediate release vasoconstrictive agents. Sustained release of vasoconstrictor agents, such as epinephrine, can achieve local tissue concentrations that are safe and effective to provide vasoconstrictor activity and to substantially prolong local anesthesia. The local circulatory bed, i.e., blood vessels, remain responsive to the vasoconstrictor agent for prolonged periods, e.g., receptor desensitization or smooth muscle fatigue or tolerance does not prevent the prolongation effect.

As for the previously discussed augmenting agents, vasoconstrictive augmenting agents can be administered before, simultaneously with or after the administration of analgesic (as may any other augmenting agent, if appropriate). In another embodiment, the vasconstrictive agent is prepared in one or separate sustained release formulations separate from the subject pharmaceutical compositions containing an pharmaceutically acceptable analgesic salt (as may any other augmenting agent, if appropriate).

Augmenting agents that are vasoconstrictor agents include, but are not limited to, catecholamines, e.g., epinephrine, norepinephrine and dopamine as well as, e.g., metaraminol, phenylephrine, methoxamine, mephentermine, methysergide, ergotamine, ergotoxine, dihydroergotamine, sumatriptan and analogs, and alpha-1 and alpha-2 adrenergic agonists, such as, e.g., clonidine, guanfacine, guanabenz and dopa (i.e., dihydroxyphenylalanine), methyldopa, ephedrine, amphetamine, methamphetamine, methylphenidate, ethylnorepinephrine, ritalin, pemoline and other sympathomimetic agents, including active metabolites, derivatives and mixtures of any of the foregoing.

The subject compositions may also include a wide range of additional materials. Stabilizing agents known in the art may be incorporated in compositions of the present invention. In certain embodiments, additives such as stabilizing agents are selected for their biocompatibility. With regard to all such additional materials, it may be important to maintain the polarity of the resulting subject composition so that the salt of an analgesic agent is at most sparingly soluble in such composition.

A composition of this invention may further contain one or more adjuvant substances, such as fillers, thickening agents or the like. For example, fillers, such as bovine serum albumin (BSA) or mouse serum albumin (MSA), may be used. Incorporation of such fillers may affect the length of the extended therapeutic effect, possibly by slowing the release rate. Other fillers known to those of skill in the art, such as carbohydrates, sugars, starches, saccharides, celluoses and polysaccharides, including mannitose and sucrose, may be used in certain embodiments in the present invention.

Buffers, acids and bases may be incorporated in the subject compositions to adjust their pH. Agents to increase the diffusion distance of agents released from the subject compositions may also be included.

Other materials known to one of skill in the art may be used to advantage to control the length, magnitude and other features of the therapeutic effect achieved by the subject compositions without departing from the spirit of the invention.

D. Use of the Pharmaceutical Compositions

In certain embodiments, the subject compositions are administered to a subject to reduce pain or treat some other disease or condition of the subject. The therapeutically effective amount of pharmaceutical composition to be administered will depend on a number of factors known to one of skill in the art, including the severity of the subject's disease or condition, the identity of the pharmaceutical composition, the mode of administration, and the like.

The pharmaceutical compositions of the present invention may be administered by various means, depending on its intended use, as is well known in the art. For example, the inventive compositions may be administered parenterally as injections (e.g., intravenous, intramuscular, epidural, or subcutaneous).

In certain embodiments, a fluid pharmaceutical composition may be especially suitable for treatment. A fluid material may be adapted for injection or instillation into a tissue mass or into an actual or potential space. A flowable material, often capable of assuming the shape of the contours of an irregular space, may be delivered to a portion of an actual or potential space to flow therefrom into a larger portion of the space. In this way, the flowable material may come to coat an entire post-operative surgical site after being inserted through an edge of an incision or after being instilled through a drain or catheter left in the surgical bed. Alternatively, if the flowable material is inserted under pressure through a device such as a needle or a catheter, it may perform hydrodissection, thus opening up a potential space and simultaneously coating the space. A flowable composition may be particularly adapted for instillation through a needle, catheter or other delivery device such as an endoscope, since its flowable characteristics allow it to reach surfaces that extend beyond the immediate reach of the delivery device. A flowable composition in a highly fluid state may be suitable for injection through needles or catheters into tissue masses, such as margins of resection sites.

Some analgesic salts such as lidocaine HCl and bupivacaine HCl have demonstrated effectiveness in alleviating tinnitus, or ringing of the ears (Weinmeister, K. P. Reg. Anesth Pain Med January-February 2000; 25(1):67-8; “Lidocaine Perfusion of the Inner Ear plus IV Lidocaine or Intractable Tinnitus,” are John J. Shea and Xianxi Ge, American Otological Society meeting, May 13-14, 2000). Treatment with analgesic such as lidocaine or bupivacaine over an extended period of time in the ear would avoid difficulties associated with frequent injections and side effects which may result from sustained systemic levels of analgesic. For the treatment of tinnitus, the compositions are used to ameliorate the false perception of sound, such as a ringing sound, in a patient, in some cases resulting in an improvement in hearing. Tests for efficacy may be performed in humans after obtaining data indicative of the compound's safety, or an animal model may be employed (Zhang, et al. Neurosci Lett 1998, 250(3), 197-200).

In certain embodiments, the oil of the present invention, upon contact with body fluids, may undergo gradual degradation. The life of a composition in vivo depends among other things, upon its molecular weight and biostability. In general, the greater the molecular weight of the oil, the greater the biostability and the slower any biodegradation will be.

In the event that the extended therapeutic effect realized by the subject compositions is attributable to a sustained release of the analgesic agent, as hypothesized above, the inventive compositions may be characterized by the release kinetics and type. For example, slow release may result in prolonged delivery (over, say 1 to about 2,000 hours, or alternatively about 2 to about 800 hours) of effective amounts (e.g., about 0.0001 mg/kg/hour to about 100 mg/kg/hour) of the analgesic agent (as the salt or the neutral form) or any other material incorporated in the oil.

The release rate of any incorporated material may also be characterized by the amount of such material released per day per mg of the oil. For example, in certain embodiments, the release rate may vary from about 1 ng or less of any incorporated material per day per mg of the oil to about 500 or more μg/day.mg. Alternatively, the release rate may be about 0.05, 0.5, 5, 10, 25, 50, 75, 100, 125, 150, 175, 200, 250, 300, 350, 400, 450, or 500 μg/day.mg. In still other embodiments, the release rate of any incorporated material may be 10,000 ng/day.mg or even higher.

In another aspect, the rate of release of any material from any oil of the present invention may be presented as the half-life of such material in the such an oil.

In addition to the embodiment involving protocols for in vitro determination of release rates, in vivo protocols, whereby in certain instances release rates for oils may be determined in vivo, are also contemplated by the present invention. Other assays useful for determining the release of any material from the oils of the present system are known in the art.

4. Dosages and Formulations of the Subject Compositions

In certain embodiments, the subject pharmaceutical compositions will incorporate the analgesic salt to be delivered in an amount sufficient to deliver to a patient a therapeutically effective amount thereof or such other material as part of a prophylactic or therapeutic treatment, which may extend for a period of time. The amount of the salt of an analgesic in the pharmaceutical composition will depend on absorption, inactivation, and excretion rates of the agent as well as the release rate of the compound from the oil. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Formulations useful in the methods of the present invention include those suitable for a variety of modes of administration, including parenteral administration. In general, the subject compositions will not require any additional formulation before administration. However, if necessary, formulation technology known to one of skill in the art may be used to formulate the subject compositions, provided that such formulation does not materially interfere with the desired therapeutic effect over an extended period of time.

The pharmaceutical compositions or formulations thereof, as the case may be, may conveniently be presented in unit dosage form and may be prepared by any methods well known in the art of pharmacy. The subject compositions may be administered once, or may be divided into a number of smaller doses to be administered at varying intervals of time, depending in part on length of the desired therapeutic effect of the subject composition and the desired dosage.

Certain pharmaceutical compositions of this invention suitable for parenteral administration and other modes of administration comprise the composition of the present invention in combination with one or more pharmaceutically-acceptable dispersions, suspensions or emulsions, or sterile powders which may be reconstituted into sterile injectable solutions or dispersions just prior to use, which may contain antioxidants, buffers, bacteriostats, or suspending or thickening agents. In certain of these cases, the formulary may involve the use an oil as defined herein. Notably, certain of the embodiments of the subject compositions are not intended to be administered topically.

5. Assays for Measuring Analgesic Effect

A variety of techniques may be used to measure analgesic effects of subject compositions, e.g., by evaluating the responsiveness of a subject, such as a rat or mouse, to a stimulus that normally provokes a response indicative of a painfuil sensation.

Rat Formalin Test. The rat formalin test is an in vivo test of analgesic potency. This test reflects several levels of processing of nociceptive information in the spinal cord. Protracted sensory input generated by the noxious stimulus employed in this test (formalin in the paw) has been shown to induce an acute pain response phase (phase 1) followed by a second phase (phase 2). This second phase is thought to represent a state of facilitated processing evoked by the afferent input present during phase 1 and to involve release of at least two substances, glutamate and a tachykinin, based on other pharmacological evidence (Yamamoto and Yaksh, Pain November 1993 55(2):227-33; Pain July 1993 54(1):79-84; Pain December 1993 51 (3):329-34; Anesthesiology October 1992 77(4):757-63; Life Sci. 1991 49(26):1955-63).

In the rat formalin test, a standard dose of formalin is injected into the rat paw, and flexions of the paw are quantitated over the following 60-minute period. A biphasic response pattern is typically observed, with numerous responses observed during the period 5 min. after injection (Phase 1) and a second phase (Phase 2) which occurs during the period about 10-60 minutes following injection, in which the mean number of flinches per minute is recorded as a function of time. Quantitation of responses during each phase is made by calculation of area under the curve of flinches/min.

Randall-Selitto Test. As described in Arch. Int. Pharmacodyn. Ther. 111, 409 (1957)), oedema can be induced in a rat's hind paw by injecting 0.1 ml of a 20% baker's yeast suspension, carrageenan, or other suitable substance, the oedema causing pronounced mechanohyperalgesia after 4 hours. Pain is then produced by applying increasing pressure (0-450 g/mm²) with a punch (0.2 mm point diameter) or other analgesiometer on the rat's inflamed hind paw. The pressure at which the rat produces a vocalisation reaction is then measured. Animals which produce no vocalisation up to the maximum permitted pressure are deemed to have complete pain relief. The test results are stated as MPE (maximum possible effect) in % in accordance with the formula: 100×(Vₜ−V₀)/(Vₘₐₓ−V₀) where Vₜ is the value measured after administration of the test substance; V₀ is the value measured before administration of the test substance, and Vₘₐₓ is the maximum value.

Hot plate test. The hot plate test (J. Pharmacol. Exp. Ther. 133, 400 (1961)) can be used to determine effectiveness of a subject composition in the event of acute, non-inflammatory, thermal stimulus. For example, rats can be gently held by the body while the plantar aspect of the paw is placed on a hot plate. The baseline (control) latency for the rat to withdraw its paw from the hot-plate (56° C.) may be determined prior to administration of an analgesic composition around the sciatic nerve. A syringe may be used to inject the composition around the sciatic nerve. Thereafter, paw withdrawal latencies are assessed. A 12 sec time limit may be employed in order to prevent damage to the paw.

Pressure Test. Analgesic effects of drugs can be evaluated using the generally accepted paw pressure test as described in C. Stein, Pharm. Biochem. Behavior, 31:445-451 (1988). The animal is gently restrained under paper wadding and incremental pressure applied via a wedge-shaped blunt piston onto an area of 1.75 mm² of the dorsal surface of the hindpaw by means of a commercially available automated gauge. The pressure required to elicit paw withdrawal (PPT) is determined. Three consecutive trials, separated by 10 sec., may be conducted and the average calculated. The same procedure can be performed on an untreated paw as a control; the sequence of paws can be altered between subjects to reduce “order” effects.

Exemplification

The invention now being generally described, it will be more readily understood by reference to the following examples which are included merely for purposes of illustration of certain aspects and embodiments of the present invention, and are not intended to limit the invention.

### EXAMPLE 1

Preparation of Lidocaine HCl

An appropriate amount of lidocaine HCl (USP) will be weighed into a clean container. A sieve with pore opening of 75 μm will be used to sieve the lidocaine HCl powder using horizontal shaking. Alternatively, lidocaine HCl (USP) may be added to a Fitzpatrick mill and milled for 2-3 minutes. The fraction that is less than 75 μm in dimension will be collected in a clean high density polypropylene container. The lidocaine HCL will be added to a clean serum bottle, the bottle sealed with an appropriate rubber stopper, crimped, and the analgesic salt sterilized by treating the bottle with Gamma irradiation.

### EXAMPLE 2

Preparation of Lidocaine HCl in Sesame Oil

Sterile sesame oil (Super Refined Sesame Oil from Croda, Inc., Part Number SSMEOL) will be withdrawn with a syringe and added to a bottle containing the sterile lidocaine HCl prepared as described in Example 1 to make a 50 mg/ml lidocaine HCl mixture in sesame oil. The mixture will be shaken to form a homogenous suspension/mixture.

### EXAMPLE 3

Plasma Studies

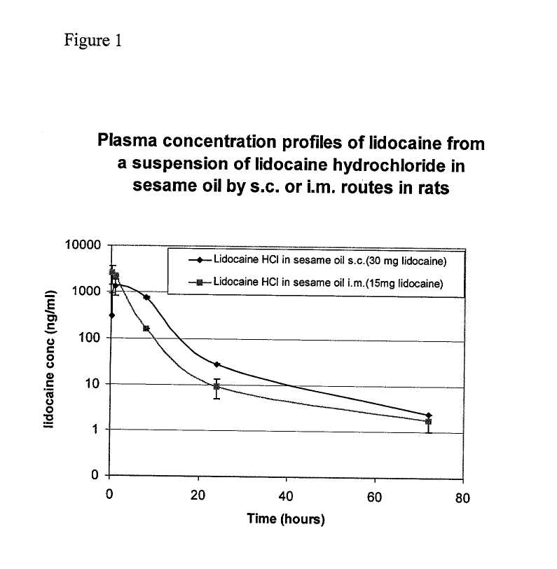

The pharmaceutical composition prepared in Example 2 was administered to five male Sprague-Hawley rats. The route of administration was subcutaneous; the location was in the animals' flanks. Blood samples were taken subsequently and plasma prepared. Plasma concentration of lidocaine following s.c (30 mg lidocaine) or i.m. (15 mg lidocaine) administration of lidocaine HCl/sesame oil formulation was determined by LC-MS/MS. FIG. 1 shows the plasma concentrations of lidocaine over time.

### EXAMPLE 4

Toxicology Study

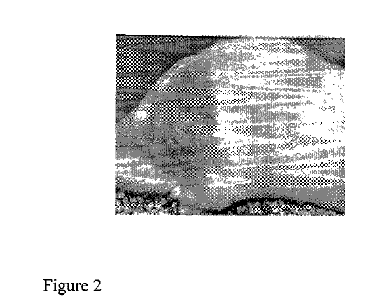

Initial results from a pilot biocompatibility study using the pharmaceutical composition of Example 2 are described here. Sprague-Hawley rats were dosed subcutaneously at a dose volume of 1 ml at a dose volume of 1 ml suspension, which contains 10 mg of lidocaine HCl. No abnormal observations were observed at the injection site. (See FIG. 2) Lesion severity was deemed by pathology standards to be low.

### EXAMPLE 5

Therapeutic Effect in Rats

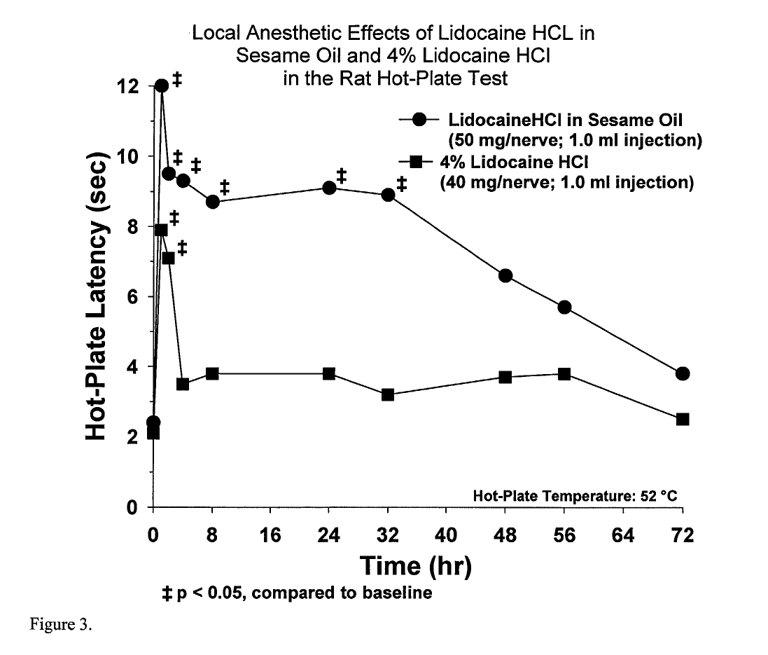

This Example shows in vivo analgesia in rats for the composition of lidocaine HCl and sesame oil. For comparative purposes, 4% lidocaine HCl in saline was used in a parallel test. Groups of six male Sprague-Hawley rats were used for each dose. For injection of one dose of the pharmaceutical composition from Example 2 (50 mg/nerve; 1.0 ml injection), the rats were briefly anesthetized with isoflurane to prevent voluntary skeletal muscle contraction during the nerve stimulation procedure. To inject local anesthetics, a sterile 22-gauge STIMEX-4 parylene coated needle (Becton Dickenson, Franklin Lakes, N.J.) was inserted into a 1½ inch 18-gauge needle (Becton Dickenson). The STIMEX-4 needles are coated with parylene to prevent electrical conduction throughout the needle, except at the tip that is un-coated. The fur was depilated at the site of injection, cleansed with sterile cotton swabs saturated with 10% providone iodine and rinsed with cotton swabs saturated with sterile isotonic saline. The surface skin was gently punctured with an 18-gauge needle in order to allow the 18-gauge/STIMEX-4 needle combination to be inserted into the tissue surrounding the nerve. The 18-gauge/STIMEX needle—with attached negative electrode—was inserted through the skin, between the greater trochanter of the femur and the ischial tuberosity of the pelvis. The positive electrode was placed on the forepaw. Electrical impulses (Digi Stim II®: <0.9 mA, and 1 Hz) delivered to the sciatic nerve caused hind limb flexion, whereas misplacement of the needle in skeletal or connective tissue failed to stimulate the hind limb. In fact, very close placement led to Digi Stim readings of <0.2 mA. Upon placement of the 18-gauge/STIMEX-4 needle combination, the STIMEX-4 needle was removed while leaving the 18-gauge needle in place near the sciatic nerve. Just before inserting the 18-gauge/STIMEX-4 needle into the animal, the composition was briefly suspended by vortexing, and then drawn up into a 1.0 ml or 3.0 ml disposable syringe. Syringe volumes were increased an additional 0.07 ml (i.e. 0.5 ml injection volume+0.07 ml=0.57 ml; 1.5 ml=1.57 ml, 3.0 ml=3.07 ml), since this represents the dead space of the 18-gauge needle. Thus, the injection of 0.57 ml resulted in 0.5 ml of composition deposited around the sciatic nerve. Results of these experiments over time are shown in FIG. 3.

### EXAMPLE 6

Pharmacology Study

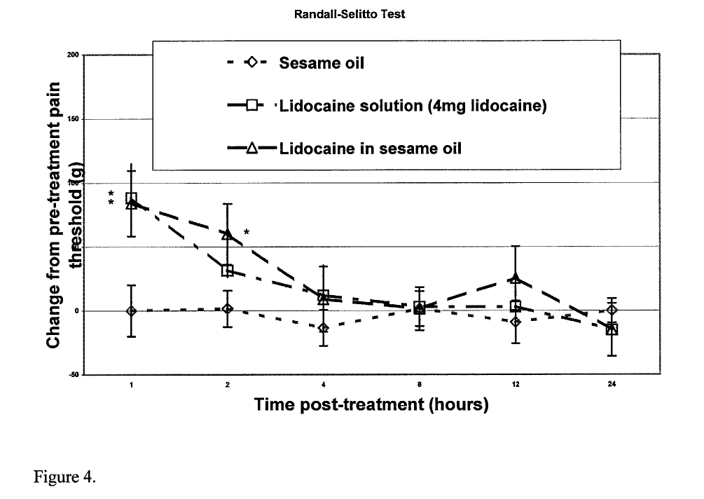

Using 4 mg lidocaine HCl with an injection volume of 80-100 uL, and N=8 rats per formulation, the Randall-Selitto assay was performed. FIG. 4 shows the change from pre-treatment pain threshold vs. time post treatment in hours.

### REFERNCES

All publications and patents mentioned herein, including those items listed below, are hereby incorporated by reference in their entirity as if each individual publication or patent was specifically and individually indicated to be incorporated by reference. In case of conflict, the present application, including any definitions herein, will control.

U.S. Pat. Nos. 6,063,762; 5,931,809; 4,954,298; 4,978,332; 5,439,686; 5,573,781; 5,622,993; 5,747,060; 5,853,732; 5,993,836; 6,214,387; 6,217,911; 5,962,016; 5,618,563; 5,993,836; 5,853,732; 5,622,993; 6,197,331; 6,166,173; 6,165,500; 6,159,498.

WO 94/20144; JP 8259464; DE 2507417; WO 95/09613; WO 94/05265; WO 94/20144; WO 97/44016; WO 97/49391; WO 00/64437.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A flowable pharmaceutical composition, comprising: a biocompatible oil and a therapeutically effective amount of a pharmaceutically acceptable salt of an analgesic agent, wherein said salt of said analgesic agent is at most sparingly soluble in said pharmaceutical composition.

2. The flowable pharmaceutical composition of claim 1, wherein said biocompatible oil is a vegetable oil.

3. The flowable pharmaceutical composition of claim 2, wherein said biocompatible oil is one of the following: canola oil, castor oil, coconut oil, corn oil, cottonseed oil, olive oil, palm oil, peanut oil, rapeseed oil, soy bean oil, safflower oil, sesame oil, soybean oil, sunflower oil, and mixtures thereof.

4. The flowable pharmaceutical composition of claim 1, wherein said biocompatible oil is sesame oil.

5. The flowable pharmaceutical composition of claim 1, wherein said biocompatible oil has a viscosity below about 140 cSt at 20° C.

6. The flowable pharmaceutical composition of claim 1, wherein said pharmaceutical composition has a viscosity below about 90 cSt at 20° C.

7. The flowable pharmaceutical composition of claim 1, wherein said biocompatible oil has a viscosity above about 45 cSt at 20° C.

8. The flowable pharmaceutical composition of claim 1, wherein said pharmaceutical composition has a viscosity between about 60 and 90 cSt at 20° C.

9. The flowable pharmaceutical composition of claim 1, wherein said pharmaceutical composition is flowable at room temperature.

10. The flowable pharmaceutical composition of claim 1, wherein said biocompatible oil has a dielectric constant below about 20.

11. The flowable pharmaceutical composition of claim 1, wherein said pharmaceutical composition has a dielectric constant below about 20.

12. The flowable pharmaceutical composition of claim 10, wherein said biocompatible oil has a dielectric constant below about 5.

13. The flowable pharmaceutical composition of claim 1, wherein all biocompatible oils in said flowable pharmaceutical composition comprise at least about 33% by weight of said flowable pharmaceutical composition.

14. The flowable pharmaceutical composition of claim 13, wherein all biocompatible oils in said flowable pharmaceutical composition comprise at least about 50% by weight of said flowable pharmaceutical composition.

15. The flowable pharmaceutical composition of claim 14, wherein all biocompatible oils in said flowable pharmaceutical composition comprise at least about 75% by weight of said flowable pharmaceutical composition.

16. The flowable pharmaceutical composition of claim 14, wherein all biocompatible oils in said flowable pharmaceutical composition is at least about 90% by weight of said flowable pharmaceutical composition.

17. The flowable pharmaceutical composition of claim 1, wherein all biocompatible oils in said flowable pharmaceutical composition comprise at least about 50% by weight of said flowable pharmaceutical composition other than all pharmaceutically acceptable salts of analgesic agents in said pharmaceutical composition.

18. The flowable pharmaceutical composition of claim 17, wherein all biocompatible oils in said flowable pharmaceutical composition comprise at least about 95% by weight of said flowable pharmaceutical composition other than all pharmaceutically acceptable salts of analgesic agents in said pharmaceutical composition.

19. The flowable pharmaceutical composition of claim 1, wherein said salt of said analgesic agent comprises at least about 2% by weight of said flowable pharmaceutical composition.

20. The flowable pharmaceutical composition of claim 19, wherein said salt of said analgesic agent comprises at least about 3% and no more than about 80% by weight of said flowable pharmaceutical composition.

21. The flowable pharmaceutical composition of claim 20, wherein all biocompatible oils in said flowable pharmaceutical composition comprise at least about 50% by weight of said flowable pharmaceutical composition.

22. The flowable pharmaceutical composition of claim 19, wherein said salt of said analgesic agent comprises at least about 4% and no more than about 67% by weight of said flowable pharmaceutical composition.

23. The flowable pharmaceutical composition of claim 22, wherein all biocompatible oils in said flowable pharmaceutical composition comprise at least about 70% by weight of said flowable pharmaceutical composition.

24. The flowable pharmaceutical composition of claim 20, wherein said salt of said analgesic agent comprises at least about 10% by weight of said flowable pharmaceutical composition.

25. The flowable pharmaceutical composition of claim 24, wherein said salt of said analgesic agent comprises at least about 40% by weight of said flowable pharmaceutical composition.

26. The flowable pharmaceutical composition of claim 20, wherein said pharmaceutically acceptable salt of said analgesic agent is an analgesic agent and an inorganic or organic acid addition salt to said analgesic agent.

27. The flowable pharmaceutical composition of claim 1, wherein said analgesic agent is a caine analgesic.

28. The flowable pharmaceutical composition of claim 27, wherein all biocompatible oils in said flowable pharmaceutical composition comprise at least about 50% by weight of said flowable pharmaceutical composition.

29. The flowable pharmaceutical composition of claim 28, wherein all biocompatible oils in said flowable pharmaceutical composition comprise at least about 85% by weight of said flowable pharmaceutical composition.

30. The flowable pharmaceutical composition of claim 1, wherein said salt of said analgesic agent is a pharmaceutically acceptable salt of lidocaine.

31. The flowable pharmaceutical composition of claim 30, wherein said salt of said analgesic agent is lidocaine HCl.

32. The flowable pharmaceutical composition of claim 31, wherein said biocompatible oil is a vegetable oil.

33. The flowable pharmaceutical composition of claim 31, wherein said biocompatible oil is sesame oil.

34. A kit for treating a disease or condition of a subject, comprising (a) any of the flowable pharmaceutical compositions claimed above, and (b) instructions for combining said biocompatible oil and said salt of said analgesic agent to form a pharmaceutical composition and for administering said flowable pharmaceutical composition to a subject.

35. The kit of claim 34, wherein said disease or condition is pain.

36. The kit of claim 34, wherein said disease or condition is tinnitus.

37. The kit of claim 34, wherein said instructions further provide for parenteral administration of said flowable pharmaceutical composition.

38. The kit of claim 34, wherein said instructions further provide for administration by injection of said flowable pharmaceutical composition.

39. A biocompatible pharmaceutical composition, comprising a biocompatible oil, at least about 2% by weight of a pharmaceutically acceptable salt of an analgesic agent, and no more than 10% by weight of a solvent in which said pharmaceutically acceptable salt of said analgesic agent is at least slightly soluble.

40. The pharmaceutical composition of claim 39, wherein said solvent comprises no more than 5% by weight of said pharmaceutical composition.

41. A biocompatible pharmaceutical composition, consisting essentially of a biocompatible oil and at least about 1% by weight of a pharmaceutically acceptable salt of an analgesic agent.

42. A method for treating a disease or condition of a subject, comprising administering parenterally to a subject a pharmaceutical composition comprising (a) a biocompatible oil, and (b) a therapeutically effective amount of a pharmaceutically acceptable salt of an analgesic agent, wherein said salt of said analgesic agent is at most sparingly soluble in said pharmaceutical composition and said pharmaceutical composition is flowable at the body temperature of said subject.

43. The method of claim 42, wherein said pharmaceutical composition is administered subcutaneously or intramuscularly.

44. The method of claim 42, wherein said pharmaceutical composition is administered by injection.

45. The method of claim 42, wherein administration of said pharmaceutical composition to a rat results in doubling of a paw withdrawal latency time in a hot plate test for at least about 36 hours.

46. The method of claim 42, wherein administration of said pharmaceutical composition to a rat results in doubling of a paw withdrawal latency time in a hot plate test for at least about 3 days.

47. The method of claims 42, wherein said biocompatible oil has a viscosity allowing said pharmaceutical composition to be administered by injection at room temperature.

48. The method of claim 42, wherein said pharmaceutical composition releases a therapeutically effective amount of said analgesic agent over about at least about 24 hours upon said administration.

49. The method of claim 42, wherein said pharmaceutical composition releases a therapeutically effective amount of said analgesic agent over about at least about four days upon said administration.

50. The method of claim 42, wherein said disease or condition is pain.

51. The method of claim 50, wherein said pain is treated or alleviated for a period of at least about twelve hours after administration of said pharmaceutical composition.

52. The method of claim 50, wherein said pain is treated or alleviated for a period of at least about one day after administration of said pharmaceutical composition.

53. The method of claim 50, wherein said pain is treated or alleviated for a period of at least about three days after administration of said pharmaceutical composition.

54. The method of claim 42, wherein said disease or condition is tinnitus.

55. A method for treating pain in a subject, comprising administering parenterally to a subject a flowable pharmaceutical composition comprising (a) a biocompatible oil, and (b) a pharmaceutically acceptable salt of an analgesic agent, wherein said pharmaceutically acceptable sale of said analgesic agent comprises at least about 2% by weight of said pharmaceutical composition.

56. The method of claim 55, wherein said pharmaceutical composition releases a therapeutically effective amount of said analgesic agent over about at least about 24 hours upon said administration.
